(19) United States
(12) Patent Application Publication
(10) Pub. No.: US 2025/0325314 A1
(43) Pub. Date: Oct. 23, 2025

(54) BONE FASTENER WITH A DIVERGING CANNULATION

(71) Applicants: Harald KINZNER, Geroldswil (CH); IGNITE-CONCEPTS GMBH, Attiswil (CH)

(72) Inventors: Harald KINZNER, Geroldswil (CH); Tom OVERES, Attiswil (CH); Valeska MELDE, Maintal (DE); Hans Jürgen WACHTER, Groß-Zimmern (DE)

(21) Appl. No.: 18/870,996

(22) PCT Filed: Jun. 2, 2023
(86) PCT No.: PCT/EP2023/064875

§ 371 Date: Dec. 2, 2024

(30) Foreign Application Priority Data
Jun. 3, 2022 (CH) 000685/2022

(51) Int. Cl. A61B 17/86; A61L 31/02; B33Y 80/00; C22C 16/00

(52) U.S. Cl. CPC A61B 17/864; A61B 17/861; A61B 17/866; A61L 31/022; B33Y 80/00; C22C 16/00; A61L 2430/02

(57) ABSTRACT

A cannulated bone fastener (10) for engagement into a target bone and/or target bone fragment is provided. According to one embodiment. the cannulated bone fastener (10) comprises an elongated shaft (11) extending between a bone fastener tip or tip section and a bone fastener head or head section. a central cannulation (17) having a cannulation entry with a first cross-sectional area (A1) at the shaft tip section, and a cannulation exit with a second cross-sectional area (A2) at the head section, and wherein the first cross-sectional area (A1) is different from the second cross-sectional area (A2), and wherein the surface roughness of the cannulation wall is at most 25 micrometres.

### TECHNICAL FIELD OF THE INVENTION

The present invention relates to a cannulated bone fastener, such as a pedicle screw or other bone screw, which is intended to be inserted into a bone over a guide wire, such as for example a Kirschner wire. In one aspect of the invention, the cannulated bone fastener comprises an at least partly conical, tapered or diverging cannulation extending along a larger part of the bone fastener length and so providing an increasing clearance between the outer diameter of the guide wire and the inner diameter of the bone fastener cannulation. As a result, the potential direct or indirect contact area between the bone fastener and the guide wire is reduced and unwanted translation or migration of the guide wire during insertion of the bone fastener is inhibited.

### BACKGROUND OF THE INVENTION

In orthopaedic surgeries, bone fasteners, such as bone screws, are often placed to a target site to stabilise two or more bones or bone portions in relation to each other.

For example, in case a long bone is broken, bone fasteners are combined with a trauma plate to bridge the fracture, and so stabilise the bone portions for healing or bone fusion.

In one other example, in case a spinal column is degenerated, the spinal column needs to be realigned and stabilised by a pedicle screw-based posterior stabilisation system.

Bone fasteners, such as pedicle screws, are placed into the affected vertebral bodies through the pedicle bone and later rigidly connected to a rod.

In one other example, in case a proximal femoral neck is broken, hip screws are placed through the neck into the femoral head to keep the femoral head in place. The stabilised construct will allow the femoral neck to fuse and heal.

In many cases when bone fasteners or bone screws are placed into a target bone, prior to the placement of the bone screw, in a first step, a guide wire is placed. In a following step, the bone screw is placed over the guide wire.

For this purpose, typically, the bone fastener comprises a cylindrical central channel or cannulation extending through the bone fastener along its length, and which is minimally oversized in relation to the guide wire.

A reason for the use of a guide wire is to define the intended trajectory of the bone screw without larger bone removal. In case the trajectory needs to be adapted by replacement of the guide wire, the stability of the bone screw, which is placed in a next step, will not be compromised.

Another reason for the use of a thin guide wire is better visualisation of the trajectory when more bone screws are placed near each other.

One other reason for the use of a guide wire is ease of placement of a bone screw in a minimal invasive manner through small skin incisions and incisions through soft tissue, such as muscles, etc. For example, in minimally invasive posterior stabilisation procedures, the screw trajectory is prepared using an awl or probe with a central channel. Before removal of the awl, a thin guide wire is placed into the awl channel. This guide wire marks the direction of the trajectory of the pedicle screw and the entry point into the bone. After removal of the awl, a pedicle screw or bone fastener assembly is placed over the guide wire and screwed into the target bone. Due to this technique, only little visualisation of the operation site is necessary. After placement of the implant, the guide wire is removed.

The use of a guide wire and a cannulated bone screw sized and shaped to engage over the guide wire brings many advantages during surgery. Still, the use of a guide wire also incorporates a clinical risk.

When a screw is placed over a guide wire, friction may be present between the outer surface area of the guide wire and the inner surface area of the cannulation which extends through the bone screw. Due to this friction, upon insertion of the bone screw into the target bone, the guide wire may be pushed forward into or through the target bone. This friction may occur due to different causes. The friction may be caused by tissue particles between the guide wire and the bone screw. The friction may be caused by a small bend or not-straightness of the thin guide wire, which is unwantedly created during the insertion of the guide wire into the bone. The friction may be caused by a non-homogenous bone density, which may cause the bone screw to deviate from the defined trajectory.

Another cause of friction may be a too high surface roughness of the cannulation.

Especially in spinal surgery, an unnoticed and unwanted translation of a guide wire may be dangerous for a patient. If upon insertion of a pedicle screw over a guide wire, a wandering or translation of the guide wire is caused, the guide wire may penetrate through the anterior wall of the vertebral body towards critical structures, such as major veins and arteries. A penetration or damage of these structures can be life-endangering.

### SUMMARY OF THE INVENTION

It is an object of the present invention to overcome at least some of the problems associated with placement of a cannulated bone fastener or bone screw over a guide wire. A solution is needed that reduces the risks of an unwanted wandering or translation of a guide wire during the bone screw insertion or implantation into a target bone.

Therefore, there is a need for a cannulated bone fastener that comprises a cannulation that reduces the risk of having friction between the outer surface area of the guide wire and the inner surface area of the cannulation.

According to a first aspect of the invention, there is provided a cannulated bone fastener as recited in claim 1.

The proposed cannulated bone fastener comprises an at least partly conical, tapered or diverging cannulation extending along a larger part or a majority portion of the bone fastener length and so provides a gradually increasing clearance between the outer diameter of the guide wire and the inner diameter of the bone fastener cannulation.

The cannulated bone fastener may be made of an amorphous metal and manufactured by means of metal injection moulding.

According to a second aspect of the invention, there is provided a method of manufacturing the cannulated bone fastener as recited in claim 16.

Other aspects of the invention are recited in the dependent claims attached hereto.

### BRIEF DESCRIPTION OF THE DRAWINGS

Other features and advantages of the invention will become apparent from the following description of non-limiting example embodiments, with reference to the appended drawings, in which:

FIGS. 1A to 1D depict an example cannulated bone fastener according to a first embodiment of the present invention;

FIGS. 2A to 2C depicts the cannulated bone fastener engaged over a guidewire;

FIG. 3 depicts a variant of the cannulated bone fastener;

FIG. 4 depicts another variant of the cannulated bone fastener;

FIGS. 5A and 5B depict another variant of the cannulated bone fastener;

FIGS. 6A to 6D depict yet another variant of the cannulated bone fastener; and

FIGS. 7A to 7C depict yet another variant of the cannulated bone fastener.

### DETAILED DESCRIPTION OF THE INVENTION

The embodiments of the present invention will now be described in detail with reference to the attached figures. The embodiments are described in the context of a cannulated bone screw. When the words first and second are used to refer to different elements, it is to be understood that this does not necessarily imply or mean that the first and second elements are somehow structurally substantially different elements or that their dimensions are substantially different unless implicitly or explicitly stated. A bone fastener in this context means a structural element, which can be brought into the target bone, and forms a stable connection between target bone fragments or between a target bone and an additional implant component, such as a bone plate or a posterior rod. Most often, a bone fastener is a fastening element, such as a bone screw. Identical or corresponding functional and structural elements which appear in the different drawings are assigned the same reference numerals.

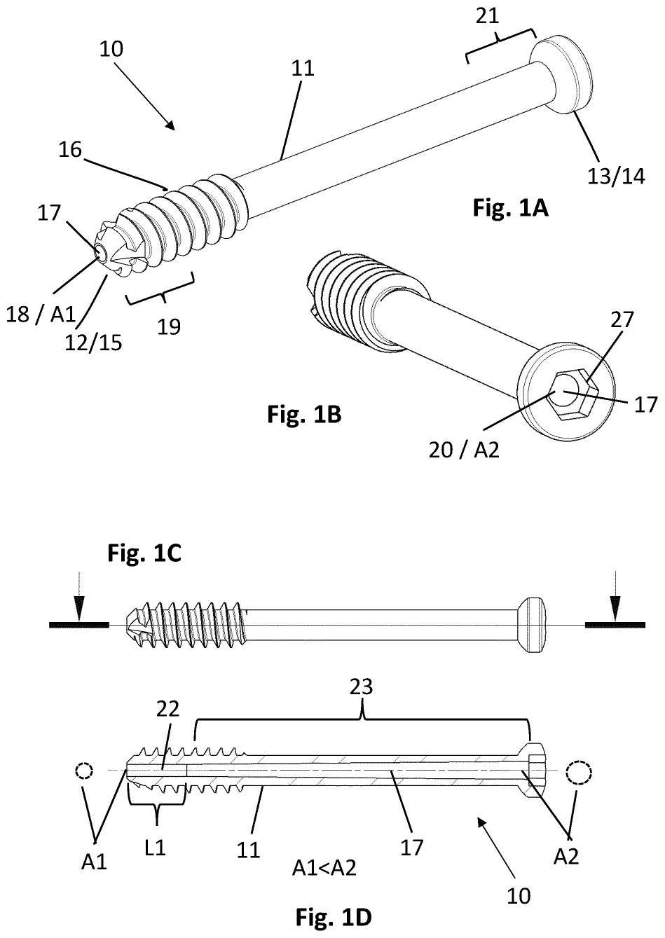

Referring to FIGS. 1A to 1D, a cannulated bone fastener 10 according to an example embodiment of the present invention is shown in perspective, orthogonal and cross-sectional views. According to the present embodiment, the cannulated bone fastener comprises an elongated shaft 11 extending between the bone fastener tip 12 and a bone fastener head 13. The head 13 ends at the proximal end 14, and the tip ends at the distal end 15. For insertion, the head 13 further comprises a drive 27 for engagement with an insertion tool, such as a screwdriver. In this example, the drive 27 starts at the proximal end 14 and extends or protrudes into the screw head. The drive shown is a hexagonal drive. Alternatively, the drive may have a different shape, such as a hexalobe drive, square drive, a Phillips drive, etc.

Moreover alternatively, the drive may protrude from the head and form an external drive. Furthermore, the drive may be formed by the head, for example as a hexagonal head. In this example, a portion of the elongated shaft comprises an external thread 16. Alternatively, the thread may extend over the entire length or substantially entire length of the shaft.

FIG. 1D shows the cannulated bone fastener in a cross-sectional view. The cannulated bone fastener comprises a central cannulation 17 having an inner wall and extending longitudinally through the bone fastener. The cannulation is configured to receive a guide wire.

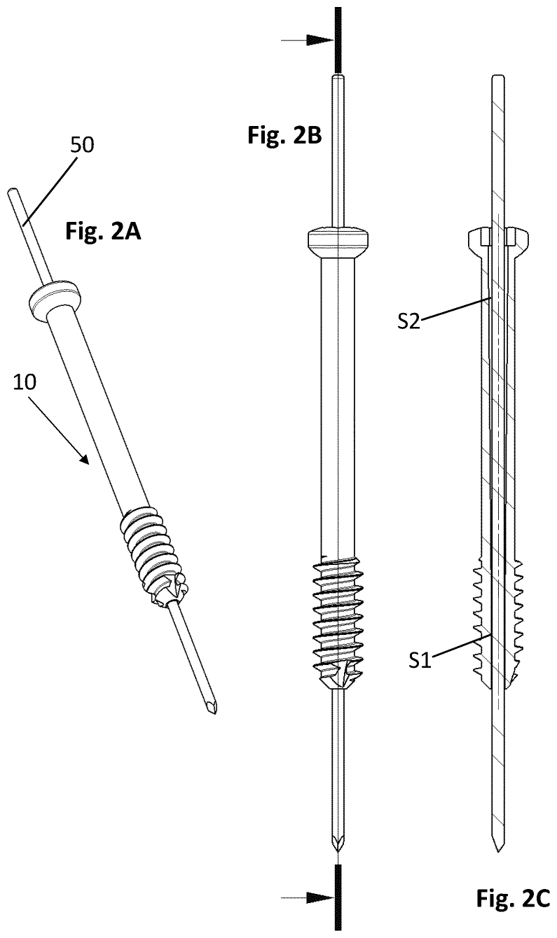

Typically, a bone fastener cannulation is a cylindrical central channel extending through the bone fastener 10 between its proximal and distal ends 14, 15, and which is minimally oversized in relation to the guide wire 50 (see FIGS. 2A to 2C). As described, a minimally oversized cylindrical channel or cannulation can cause the guide wire to jam inside the cannulation due to tissue debris, a non-straight guide wire, or a small deviation of the trajectory of the bone fastener in relation to the trajectory of the guide wire. This jamming or enlarged friction can cause the guide wire to translate during insertion of the cannulated bone fastener.

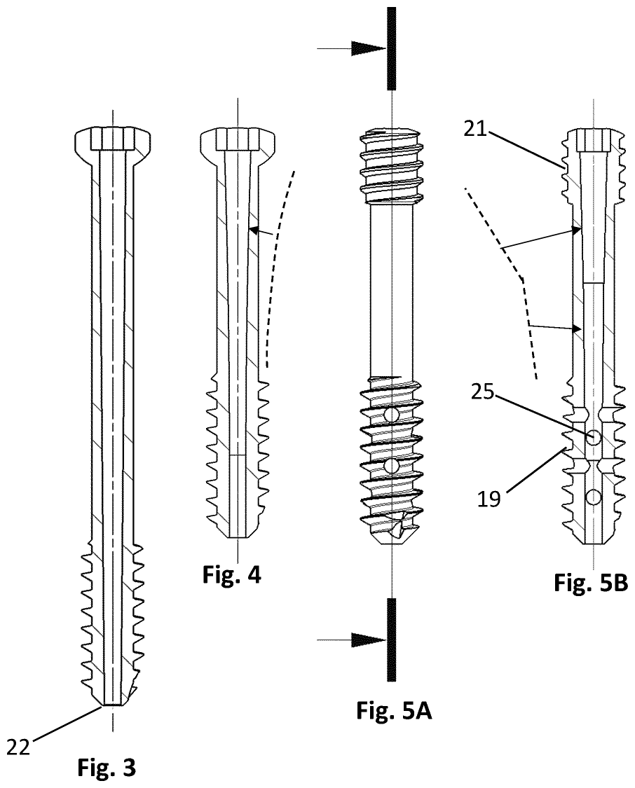

In order to overcome this problem, in this example, the bone fastener cannulation 17 (or central channel) comprises a cannulation entry 18 at the bone fastener tip 12 or tip section 19 and a cannulation exit 20 at the head section 21. The entry and exit have a first cross-sectional area A1 and a second cross-sectional area A2, respectively. The first cross-sectional area is smaller than the second cross-sectional area and is minimally oversized in relation to the guide wire it is intended to engage over. Hence, the cannulation will only provide guidance to the guide wire at the tip section 19. In this example, the bone fastener cannulation 17 comprises a first cannulation portion 22. In the present example, the first cross-sectional area is substantially constant over the first cannulation portion 22 and the first cannulation portion 22 extends over a length ‘L1’ of at least 2 mm. However, according to another example, the first cannulation portion has a length of at least 5 mm. However to another example, as depicted in FIG. 3, the first cross-sectional area A1 starts and ends at the tip, and therefore the first cannulation portion 22 has a close to zero or zero length. The difference between the cross-sectional areas implicitly generates a transition section 23 or a second cannulation portion 40 (or a first diverging cannulation portion). In the examples as shown in FIGS. 1A to 1D and 2A to 2C, the transition section 23 is configured as a substantially constantly diverging taper (i.e., a section with a non-constant cross section orthogonally to the longitudinal axis of the bone fastener). Alternatively, multiple diverging sections as shown in FIGS. 5A and 5B or a curved transition as shown in FIG. 4 may serve the same purpose. The example of FIGS. 5A and 5B further comprises a threaded head section 26. Furthermore, at the tip section 19 and/or first middle section 24, the bone fastener of FIGS. 5A and 5B comprises one or more bores, channels, fenestrations or openings 25, which are open towards the outside of the bone fastener. In some clinical cases, in a very osteoporotic bone, a bone fastener cannot provide the needed primary stability. In these cases bone cement is injected into the target bone through the cannulation and the openings. The bone cement then cures around the bone fastener providing extra stability. These fenestrations help create a homogeneous distribution of the bone cement around the bone fastener as the bone cement can be spread around the bone fastener through these fenestrations.

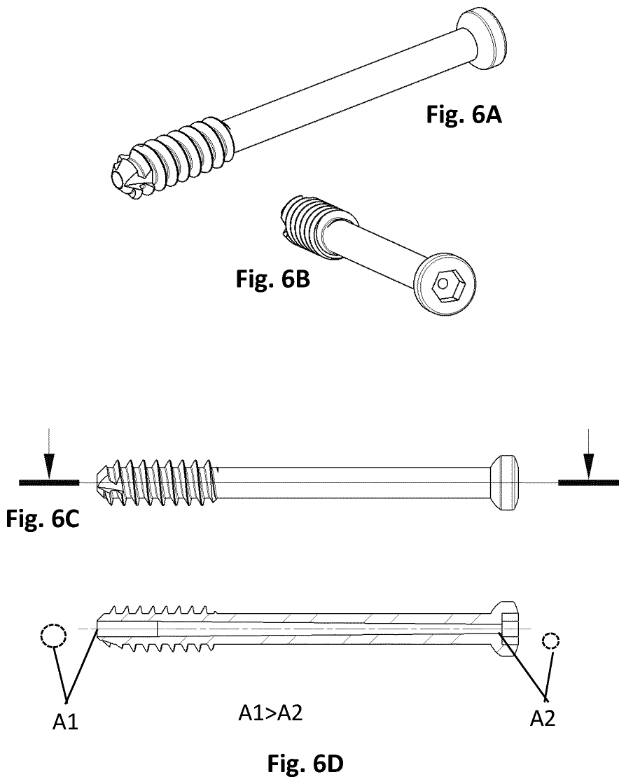

Alternatively, as shown in FIGS. 6A to 6D, the first cross-sectional area A1 may be larger than the second cross-sectional area A2.

The cannulation of the bone fastener preferably has a very smooth surface to reduce friction between the guide wire and bone fastener/cannulation. Purposefully, the surface roughness is at most 50 or 25 micrometres and preferably at most 5 micrometres and more preferably at most 1.6 micrometres. Surface roughness Ra may be measured in various ways, for example by using contact and non-contact methods, direct measurement methods, comparison methods, and/or in-process methods. Contact methods involve dragging a measurement stylus across the surface. These instruments are called profilometers. Non-contact methods include: interferometry, confocal microscopy, focus variation, structured light, electrical capacitance, electron microscopy, and photogrammetry. A smoother surface of the cannulation will reduce the risk of translation or wandering of the guide wire.

The above-described embodiment thus discloses a cannulated bone fastener 10 for engagement into a target bone and/or target bone fragment. The cannulated bone fastener 10 comprises an elongated shaft 11 extending between a bone fastener tip 12 or shaft tip section 19 and a bone fastener head 13 or head section 21, a central cannulation 17 having a cannulation entry 18 with a first cross-sectional area A1 at the shaft tip section 19, and a cannulation exit 20 with a second cross-sectional area A2 at the head section 21. The first cross-sectional area A1 is different from the second cross-sectional area A2. The surface roughness of the cannulation wall is at most 25 micrometres. The central cannulation may be diverging (non-constant cross-section) or may have one or more diverging portions.

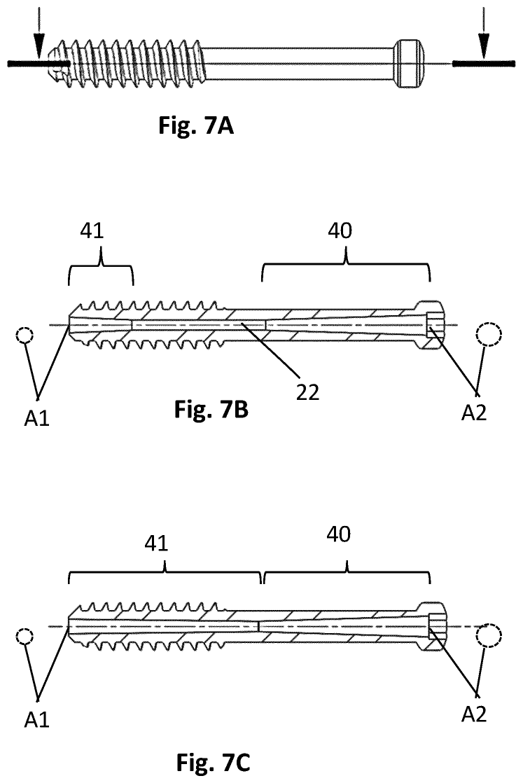

FIGS. 7A to 7C show one further variant of the cannulated bone fastener. This variant comprises two diverging cannulation portions, namely a second cannulation portion 40 (or a first diverging cannulation portion) and a third cannulation portion 41 (or a second diverging cannulation portion). In this example, the second cannulation portion diverges towards the proximal end 14, i.e., the bone fastener head, and the third cannulation portion diverges towards the distal end 15, i.e., the bone fastener tip. In this example, the second and third cannulation portions are coaxial, and optionally straight sections. In the variant shown in FIG. 7B, the first cannulation portion 22 is provided between the second and third cannulation portions to link these two portions to each other.

Referring to FIG. 7C, a variant where the first cannulation portion 22 has a close to zero or zero length is shown. It is to be noted that in the examples shown in FIGS. 7A to 7C, the first cross-sectional area A1 may be equal or substantially equal to the second cross-sectional area A2.

In one embodiment, the bone fastener is configured to be manufactured by injection moulding, wherein a liquid metal is injected into a mould, and it is made to cure rapidly. The diverging taper provides a relief angle which is advantageous for this specific manufacturing method. Moreover, in one embodiment the bone fastener is a monolithic component, which is a component that is not composed of more sub-components, which would then somehow be merged together.

Bone fasteners are commonly made of biocompatible materials, such as any one of titanium, titanium alloys, stainless steel, and cobalt chromium steel. According to the present embodiments, at least one element of the bone screw assembly and/or the accompanying rod may be made of a biocompatible amorphous metal. An amorphous metal, which is also known as metallic glass or glassy metal, is a solid metallic material, typically an alloy, with disordered atomic-scale structure. Most metals are crystalline in their solid state. This means they have a highly ordered arrangement of atoms. Amorphous metals are non-crystalline having a glass-like structure. But unlike common glasses, such as window glass, which are usually electrical insulators, amorphous metals have good electrical conductivity and they also display superconductivity at low temperatures. The amorphous metal may comprise the following first chemical composition and/or second chemical composition, the element concentration values below being given in weight percentages, namely the first chemical composition:
Zirconium (Zr): Balance;
Copper (Cu): 16% plus/minus 5%;
Nickel (Ni): 12% plus/minus 5%; and
Titanium (Ti): 3% plus 5%, minus 2%,
   and/or the second chemical composition:
Zirconium (Zr): Balance;
Copper (Cu): 24% plus/minus 5%;
Aluminium (Al): 4% plus 5%, minus 2%; and
Niob (Nb): 2% plus 5%, minus 1%.

In the above described example, the balance is the major chemical component or element. Depending on the exact percentage of the other components and possible minor impurities or other elements with a very minor weight percentage, the balance percentage gives the remaining percentage of the total 100%. The first and second compositions describe the four elements with the greatest weight percentage share.

According to an example, the amorphous metal has a limit of elasticity or elastic limit which is at least 20% higher in comparison to stainless steel. More preferably, the amorphous metal has a limit of elasticity which is at least 70% higher in comparison to stainless steel. By elastic limit is meant here the maximum stress or force per unit area within a solid material that can arise before the onset of permanent deformation. When stresses up to the elastic limit are removed, the material thus resumes its original size and shape. Stresses beyond the elastic limit cause a material to yield. The higher elasticity provides a better division of the loads comparable to other implant materials, such as titanium, titanium alloys, or cobalt chromium alloys. Amorphous metals allow for processing by means of injection moulding and additive manufacturing, i.e. three-dimensional printing. Therefore, at least one element of the bone screw assembly or the accompanying rod may be manufactured by means of injection moulding and/or additive manufacturing.

While the invention has been illustrated and described in detail in the drawings and foregoing description, such illustration and description are to be considered illustrative or exemplary and not restrictive, the invention being not limited to the disclosed embodiments. Other embodiments and variants are understood, and can be achieved by those skilled in the art when carrying out the claimed invention, based on a study of the drawings, the disclosure and the appended claims. Further embodiments or variants may be obtained by combining any of the teachings above.

In the claims, the word “comprising” or “including” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A cannulated bone fastener for engagement into a target bone and/or target bone fragment, the cannulated bone fastener comprising an elongated shaft extending between a bone fastener tip or shaft tip section and a bone fastener head or head section, a central cannulation having a cannulation entry with a first cross-sectional area at the shaft tip section, and a cannulation exit with a second cross-sectional area at the head section,
wherein the first cross-sectional area is different from the second cross-sectional area, or
wherein the central cannulation comprises a first diverging cannulation portion diverging towards a proximal end of the cannulated bone fastener, and a second diverging cannulation portion diverging towards a distal end of the cannulated bone fastener, wherein the first cross-sectional area is substantially equal to the second cross-sectional area, or the first cross-sectional area is different from the second cross-sectional area, and
wherein the surface roughness of the cannulation wall is at most 25 micrometres.

2. The cannulated bone fastener according to claim 1, wherein the first cross-sectional area is smaller than the second cross-sectional area.

3. The cannulated bone fastener according to claim 1, wherein the first cross-sectional area is larger than the second cross-sectional area.

4. The cannulated bone fastener according to claim 1, wherein the elongated shaft comprises an external thread.

5. The cannulated bone fastener according to claim 1, wherein the first cross-sectional area remains substantially constant over a first cannulation portion having a length ‘L1’ of at least 2 mm, or more specifically at least 5 mm.

6. The cannulated bone fastener according to claim 1, wherein the central cannulation comprises a transition section connecting the first cross-sectional area to the second cross-sectional area, and wherein the transition section is configured as at least one diverging taper.

7. The cannulated bone fastener according to claim 1, wherein the central cannulation comprises a transition section connecting the first cross-sectional area to the second cross-sectional area, and wherein the transition section is configured as at least one diverging curved transition.

8. The cannulated bone fastener according to claim 1, wherein the cannulated bone fastener further comprises a drive for engagement with an insertion tool, such as a screwdriver.

9. The cannulated bone fastener according to claim 1, wherein the cannulated bone fastener is made of an amorphous metal.

10. The cannulated bone fastener according to claim 1, wherein the cannulated bone fastener is manufactured by injection moulding or additive manufacturing.

11. The cannulated bone fastener according to claim 1, wherein the cannulated bone fastener has a chemical composition, given as a weight percentage, comprising:
a. Zirconium (Zr): balance;
b. Copper (Cu): 16% plus/minus 5%;
c. Nickel (Ni): 12% plus/minus 5%; and
d. Titanium (Ti): 3% plus 5%, minus 2%.

12. The cannulated bone fastener according to claim 1, wherein the cannulated bone fastener has a chemical composition, given as a weight percentage, comprising:
a. Zirconium (Zr): Balance;
b. Copper (Cu): 24% plus/minus 5%;
c. Aluminium (AI): 4% plus 5%, minus 2%; and
d. Niob (Nb): 2% plus 5%, minus 1%.

13. The cannulated bone fastener according to claim 1, wherein at least the shaft tip section comprises one or more openings leading to the central cannulation for injecting bone cement around the cannulated bone fastener.

14. The cannulated bone fastener according to claim 1, wherein the surface roughness of the cannulation wall is at most 5 micrometres, or more preferably at most 1.6 micrometres.

15. The cannulated bone fastener according to claim 1, wherein the cannulated bone fastener is a monolithic component.

16. A method of manufacturing the cannulated bone fastener according to claim 1, wherein the method comprises the step of manufacturing the bone fastener assembly by injection moulding or additive manufacturing.
